(19) United States
(12) Patent Application Publication
(10) Pub. No.: US 2013/0042873 A1
(43) Pub. Date: Feb. 21, 2013

(54) CONTRACEPTIVE DEVICE

(71) Applicants: Klaus Bechgaard, Kobenhavn O (DK); Morten Albrechtsen, Charlottenlund (DK); Galina Skladtchikova, Hellerup (DK)

(72) Inventors: Klaus Bechgaard, Kobenhavn O (DK); Morten Albrechtsen, Charlottenlund (DK); Galina Skladtchikova, Hellerup (DK)

(21) Appl. No.: 13/583,587

(22) PCT Filed: Mar. 9, 2011
(86) PCT No.: PCT/DK11/50073

§ 371 Date: Sep. 7, 2012

(30) Foreign Application Priority Data
Mar. 9, 2010 (DK) PA 2010 00187

(51) Int. Cl. A61F 6/22; C08K 3/08

(52) U.S. Cl. 128/831; 523/113

(57) ABSTRACT

A composite material for occluding a body lumen comprising at least two different components that differ from each other by their value of elastic modulus is disclosed. One component has a value of elastic modulus of less than 4 GPa, and another component has a value of elastic modulus of above 4 GPa. The component that has a higher value of elastic modulus is embedded in the component that has a lower value of elastic modulus. The composite material comprises at least one portion that is free of the component that has the higher value of elastic modulus, wherein said portion is capable of expanding in at least one direction upon application of pressure perpendicular to the expanding direction, or upon application of heat, light or a chemical or biological impact. The composite material is particular suited for manufacturing implantable contraceptive devices.

### FIELD OF INVENTION

The invention relates to the field of contraceptive and sterilization devices and more particularly to reversible contraceptive devices.

### BACKGROUND OF INVENTION

Conventional contraceptive strategies generally fall within three categories: physical barriers, drugs and surgery. While each have certain advantages, they also suffer from various drawbacks. Barriers such as condoms and diaphragms are subject to failure due to breakage and displacement. Drug strategies, such as the pill and Norplant™, which rely on artificially controlling hormone levels, suffer from known and unknown side-effects from prolonged use. Finally, surgical procedures, such as tubal ligation and vasectomy, involve the costs and attendant risks of surgery, and are frequently not reversible.

While the theoretical effectiveness of existing non-surgical contraceptive techniques, including barrier methods and hormonal therapies, is well established, the actual effectiveness of most known methods is disappointing. One reason for these disappointing results is that many of the presently available methods for inhibiting pregnancy without surgery depend upon significant user involvement. Non-compliance typically results in quite high rates of failure, and overcoming user non-compliance to improve overall efficacy has proven quite difficult.

One form of long term contraception which is less susceptible to user non-compliance is the intrauterine device (IUD). IUDs have been found to have higher rates of reliability, and are effective for a longer period of time, then most other commercially available contraceptives. Unfortunately, IUDs are also associated with serious infectious complications. For this reason, the use of IUDs has decreased dramatically. Additionally, IUDs are subject to unplanned expulsion, and are removed due to excessive pain or bleeding in a significant percentage of cases, further reducing acceptance of the IUD as a method of inhibiting pregnancy.

Commercially available options for permanent sterilization include fallopian tube ligation and vasectomy. These methods are surgical and are not available to many people in the world. It is common knowledge that fertilization occurs in the fallopian tubes where the sperm and ovum meet. Tubal ligation avoids this by surgical and complete occlusion of the fallopian tubes.

Introduction of intrafallopian contraceptive devices, for example such as described in U.S. Pat. No. 6,176,240, U.S. Pat. No. 6,763,833 or U.S. Pat. No. 7,073,504, was a significant advancement in the art. It has been proposed to transcervically introduce an expandable structure, e.g. a resilient coil into a fallopian tube which is mechanically anchored within the fallopian tube so as to inhibit conception. To provide long term contraception and/or permanent sterilization the described devices may comprise means that promote a tissue ingrowth thereby further securing closing the passage through the lumen of fallopian tubes. Transcervical introduction of the devices needs no surgical procedures, and the risks of increased bleeding, pain, and infection associated with intrauterine devices are avoided.

Still, new devices, systems, and methods that could provide a further improved non-surgical inhibition of pregnancy are highly desirable. In particular, it would be beneficial if these improved techniques increased the ease with which these contraceptive devices could be deployed, and if the improvements further enhanced the long term retention of the contraceptive device once it has been deployed. It would be further beneficial if these improved access and deployment techniques were suitable for a wide variety of physiological geometries, ideally without having to tailor the device, deployment system, or deployment method for specific individuals. It would also be highly desirable if a device having all the latter features could also provide reversible contraception.

Some or all of these advantages are provided by the devices of the present invention.

### SUMMARY OF INVENTION

The present invention is directed to a device for occluding a body lumen, such as a reproductive tract, to prevent the passage of fluid and/or cells, such as reproductive cells, through the body lumen, said device comprising
(i) an occluding member, which is a structure that can be introduced into the body lumen and then expanded within the lumen from a first smaller configuration suitable for introduction into the lumen to a second larger configuration which has transverse dimensions roughly corresponding to or slightly larger than the body lumen to facilitate securing the expanded occluding member to the wall defining the body lumen;
(ii) aids to allow expanding the occluding member within the body lumen; and
(iii) aids to facilitate sufficient securing the expanded occluding member to the wall of the body lumen to prevent the passage of fluid and cells therethrough.

The occluding member of the invention is produced from a particular composite material. Accordingly, in another aspect the invention relates to a composite material, wherein said composite material comprises at least two different components that differ from each other by the value of elastic modulus, wherein one component has a value of elastic modulus that is less than 4 GPa, and another component has a value of elastic modulus that is above 4 GPa, wherein the component that has a higher value of elastic modulus is embedded in the component that has a lower value of elastic modulus, said composite material is characterized in that it comprises at least one portion that is free of the component that has the higher value of elastic modulus, wherein said portion is capable of expanding in at least one direction upon application of pressure perpendicular to the expanding direction, or upon application of heat, light or a chemical or biological impact.

In different embodiments a device of the invention may further comprise (iv) aids to insert the device into the body lumen; (v) aids to prevent or reduce side effects associated with the insertion of the occluding member, e.g. inflammation and/or pain killer means; (vi) aids to facilitate delivering the occluding member to the right place within the body lumen, e.g. means for tracking the insertion of the occluding member, e.g. radiopaque markers for fluoroscopic placement confirmation, sonograraphic markers for ultrasound placement, or the like; (vi) aids to remove the occluding member from the body lumen, and (vii) aids to facilitate said removal, e.g. means to prevent tissue in-growth toward the occluding member. In some embodiments the device may comprise further means and components which can further strengthen its function.

### DETAILED DESCRIPTION OF INVENTION

The invention relates to a device comprising a unit that can be inserted into a body lumen, secured to the wall defining that body lumen and close, i.e. occlude, the passageway through said body lumen (said unit is herein termed “occluding member”). A body lumen may be any lumen of a body tubular structure that has an internal diameter from 0.5 to 1.5 cm, e.g. the lumen of a reproductive tract, e. g. the lumen of the fallopian tube of a female subject, lumen of vas deferens of a male subject or lumen of another reproductive tract. In one embodiment the invention relates to a contraceptive device comprising an occluding member described herein. In some embodiments, a device comprising the described herein occluding member may provide for irreversible occlusion of a body lumen, e.g. permanent contraception. In other embodiments, a device comprising the described herein occluding member may be used for reversible occlusion of a body lumen, e.g. temporary contraception.

In one embodiment the occluding member of the device may be represented by an elongated cylinder-like structure which has a length in a range from about 1 cm to about 10 cm, an outer diameter in a range from about 0.5 cm to about 2.5 cm, and two ends, end (1) and end (2). The end (1) is identified herein as the distal end of the occluding member, i.e. the end situated farthest from the point of insertion of the occluding member into the body lumen, such as e.g. the farthest from the uterus and closest to the ovary end of the fallopian tube, in case the occluding member is a part of the female sterilization device. The end (2) is identified herein as the proximal end of the occluding member, i.e. the end situated closest to the point of insertion of the occluding member into the body lumen, such as e.g. the end that is closest to the ovary end of the fallopian tube, in case the occluding member is a part of the female sterilization device.

In one embodiment, the occluding member may be a solid cylinder-shaped structure made of a composite material (the material is described in detailed below), wherein said structure has no any passage through, i.e. the structure is not a tube. Such occluding member is termed hereafter “solid occluding member”.

In another embodiment, the occluding member may be a tubular structure, i.e. the occluding member comprises a lumen or a passage spanning the occluding member from end (1) to end (2) of along its longitudinal axis, which tubular wall is made of a composite material described below. Such occluding member is termed hereafter “tubular occluding member.

In one embodiment the inner lumen of the tubular occluding member, i.e. the passageway through the occluding member, may be closed, for example one or both ends of the tubular occluding member may comprise a closure, e.g. a cap or lid, blocking the passage through the occluding member. In one embodiment of the passage may be closed reversibly, e.g. the closures at the ends of the occluding member may be removed. The closure(s) may be removed after the occluding member have been delivered and fixed to the body lumen wall at the place of delivery, and the body lumen passageway may thus be reopened again without removal of the occluding member from the lumen.

The reversible closure(s) may be removed mechanically, or they may be removed by any other impact, e.g. the closure(s) may be made of or may comprise a material that can be removed upon local application, i.e. locally on the closure, of heat, light, electric or magnetic field, or a chemical or biological impact, e.g. the closure(s) may be made of or may comprise a poly(n-propylmethylsilane-co-isopropylmethylsilane) or poly(olefin sulphone) material, such as e.g. described by Yaguchi H., & Sasaki T. (Photoinduced Depolymerization of Poly(olefin sulfone)s Possessing Photobase Generating Groups in the Side Chain. Macromolecules 2007, 40:9332-9338), or decribed by Villegas A., Olayo R., and Cervantes J. (UV Radiation-Induced Degradation of Poly(n-Propylmethylsilane-Co-Isopropylmethylsilane) in Solution. Journal of Inorganic and Organometallic Polymers, 1998, 8, No. 3), or such as described in U.S. Pat. No. 3,917,483. An occluding member having reversibly closed ends may be advantageous in cases when a temporary occlusion of a body lumen is desired, as the occlusion may be reverted any time when desired.

Alternatively, a reversible occlusion may be achieved by inserting a plug or mandrel into the internal lumen of the tubular occluding member, which plug or mandrel can be left in place to effectively blocking the passageway until the subject wishes to reverse the procedure. The plug or mandrel can be non-surgically removed by any suitable means such as conventional laparoscopic or any other instruments or means suitable to reopen the passageway. In one embodiment, the plug or mandrel may be made of a material as one of the materials described above, and, thus, it can be removed by application of a chemical impact, light, radiation, etc. A balloon dilatation catheter may be used to further expand the opening once the plug is removed.

As mentioned, in one embodiment the occluding member may be made of a solid piece of material (solid occluding member) and not comprise an inner lumen as the tubular occluding member described above. Such solid occluding member may be advantageous when a permanent occlusion of a body lumen is desired.

Both body of a solid occluding member and wall of a tubular occluding member are according to the invention formed from a composite material that comprises at least two components that differ from each other by the value of their elastic modulus, in particular the value of the Yang's modulus, wherein at least one component of the composite material has a relatively high value of elastic modulus, i.e. it has a value that is typical for materials that has no tendency to deform along an axis when opposing forces are applied along that axis, and wherein at least one another component of the composite material has a relatively low value of elastic modulus, i.e. it has a value that is typical for materials that have a tendency to deform along an axis when opposing forces are applied along that axis. The term “relatively” in the present context means that values of elastic modulus of the components may vary within a wide range. A component of the composite material that has a relatively high value of elastic modulus is termed herein “stiff component” or “stiff material”. The component of the composite material that has a relatively lower value of elastic modulus is termed herein “flexible component” or “flexible material”. The flexible component of the composite material may be a material that has a value of the Yang's modulus in a range from 0.01 to below 4 GPa, such as e.g. between 0.1 and 3 GPa, for example between 0.5 and 2 GPa, etc.; the stiff component of the composite material may be a material that has a value of the Yang's modulus in a range from 4 to 200 GPa, such as from 8 to 100 GPa, for example from 10 to 80 GPa, etc.

To make a composite material of the invention, a suitable stiff material is embedded into a suitable flexible material, i.e. the composite material may be illustrates as a piece of a flexible material that comprises internal portions of a stiff material. According to the invention, the composite material comprises at least one portion that is free of stiff material, i.e. that portion comprises only flexible material.

The embedded into flexible material portions of stiff material are preferably shaped into string or fiber, strip or plate-like structures. Such string-, fiber-, strip- and plate-like structures may be typically represented by solid pieces of a stiff material, wherein said pieces have a thickness/diameter which is less than the thickness of the wall of the tubular occluding member, such as e.g. between 1 nm and 2 mm, and have a length which is equal or less than the length of the occluding member along its longitudinal axis, such e.g. between 1 cm and 10 cm. A rectangular stiff structure depending on its width is defined as strip or plate, wherein the term “strip” relates to a piece that has a width which is about 1-20% more, about 1-20% less, or sufficiently equal to the thickness of the piece, such as e.g. 80-120% of the thickness of the piece, e.g. such as e.g. between 0.8 nm and 1.8 mm. A strip-like structure shaped as a cylinder is defined herein as “string”; several single strings hold together are defined herein as “robe” or “fiber”-like structures. A piece of a stiff component of the composite material is defined herein as “plate” when the piece has a width which is about 0.1-50% less than a circular length of the outer surface of the tubular wall of the occluding member, or about 0.1-50% more than a circular length of the inner surface of the tubular wall of the occluding member, e.g. a plate may have a width of 5 mm-1 cm, in case the tubular occluding member comprising such stiff structures is a part of a device for occluding the fallopian tube of a female subject, which fallopian tube may have a diameter of 0.5-1.2 cm.

In another preferred embodiment, a stiff component may be represented by short pin-like structures or small rectangular structures, or small structure of any other shape, which have diameter/width and thickness in the same range as a diameter/width and thickness strings/strips described above, which length is significantly less than a length of said strips or strings, i.e. significantly less than the length of the wall of a tubular occluding member along its longitudinal axis, such as 2-100 times less than the thickness of the wall of the tubular occluding member.

In one embodiment the structures of stiff component are embedded into the flexible material so that they form one or more layers within the flexible component. A composite material of this kind may be illustrated by a rectangular piece of the material which surfaces are made of a flexible material and which internally comprises one or more layers of a stiff material separated with layers of the flexible material. Accordingly, the wall of a tubular occluding member produced from such layered composite material will have the same kind layered structure, wherein the layers of a flexible component are making up both inner surface of the tubular wall defining the inner lumen of the occluding member and outer surface of the tubular wall which would face the wall of a body lumen, when the occluding member is inserted into this body lumen, and wherein the layer(s) of the stiff component is(are) spanning the wall of the occluding member from end (1) to end (2) along its longitudinal axis forming thereby a stiff layer inside of said wall. In different embodiments the wall of a tubular occluding member may be made of multiple sheets of such layered composite material, e.g. one, two, three or more sheets of such composite material.

In one embodiment, the stiff component embedded into the flexible component may not form any distinct layer within flexible component of the composite material, but form an internal mesh- or net-like framework.

Both layer-and net-organized stiff material may comprise either elongated or short stiff structures (as described above), or both.

As mentioned, the wall of a tubular occluding member of the invention may vary in thickness and be from 10 nm to 2.5 mm. Accordingly, the thickness of a sheet of a composite material making up said wall may be equal or less than a thickness of the tubular occluding member wall. A net-or mesh-like framework may occupy in different embodiments 1-99% of thickness of the wall of a tubular occluding member or of volume of the body of a solid occluding member.

Multiple structures of the stiff material (as described above) embedded into the flexible material may be unbroken and each consists of one unit. Such stiff structures are termed herein “elementary stiff structures”, i.e. one string, one plate, one strip, etc. In some embodiments stiff structures, each may be composed of several identical or similar elementary stiff structures, i.e. each string may consist of two or more identical or similar shorter strings which are interconnected to form one elongated string structure with portions of a flexible material. Such “broken” stiff structures are termed herein “complex stiff structures”. The interconnecting flexible material of complex stiff structures may be the same flexible material as one in which the complex stiff structures are embedded to form a composite material, or it may be another flexible material, e.g. a material that has another value of the Yang's modulus. Accordingly, one complex string (or a strip, plate, etc.) may comprise interconnected two or more shorter elementary stings (or strips, plates, etc.) which are of the same or similar diameter (or thickness and width), made of the same or similar material, e.g. same polymer, same metal, and have the same or similar value of elastic modulus. In one embodiment, elongated elementary strings, or shorter elementary strings of a complex stiff structure may comprise one or more hinge-like arrangements along their longitudinal axis. Such hinge-like arrangements may serve in some embodiments as means facilitating expansion of particular areas of the occluding member in the place of insertion.

As mentioned, multiple stiff strings, strips or plates may be embedded into a flexible component to form a layer within this flexible component. The thickness of such layer will be defined by the thickness/diameter of a single stiff structure, i.e. a single string, strip or plate, that makes up this layer. Thus, a thickness of a single stiff layer may vary in different embodiments, such as from 1 nm to about 2.5 mm, for example it may be 0.1-99% of the thickness of the wall of the tubular member. The thickness of flexible layers making up the surfaces of the wall of the tubular occluding member and layers the between the stiff layers of a composite material comprising more than one stiff layer may also vary as from 1 nm to around 2.5 mm according to the thickness of the stiff layer.

In one preferred embodiment, stiff structures of the composite material used for production of an occluding member are complex stiff structures, i.e. each embedded stiff structure is composed of at least two identical or similar stiff components, e.g. two strings of the same or similar diameter made of the same or similar material (e.g. a material having the same or similar value of elastic modulus), identical or different length, which are interconnected with a portion of a flexible material, wherein the total length of said two strings including the portion of the flexible material is equal to or slightly (1-20%) shorter than the total length of the occluding member. In one embodiment the occluding member is a solid occluding member, in another embodiment the occluding member is a tubular occluding member.

In one embodiment, complex stiff structures may form at least one layer within the wall of a tubular occluding member. A stiff layer comprising multiple complex stiff structures, such as strings or strips, may according to the invention be organized so that the interconnecting portions of flexible material of every complex stiff structure are aligned to each other so that the stiff layer have at least one portion which is consists of only the flexible material, i.e. the stiff material free. Accordingly, the wall of a tubular occluding member comprising such stiff layer also comprises a portion which is free of the stiff material and consists of only the flexible material. In embodiment when each complex stiff structure of the stiff layer comprises three or more elementary stiff structures interconnected with two or more portions of a flexible material, the elementary stiff structures are organized in the layer so that the layer comprises two or more portions that are free of the stiff material and consists of only the flexible material. Accordingly, the wall of a tubular occluding member comprising such stiff layer also comprises two or more portions which are free of the stiff material and consist of only the flexible material. Preferably, each complex stiff structure of the stiff layer within the wall of a tubular occluding member consists of three elementary stiff structures and two interconnecting portions of a flexible material. Preferably, the portions of the flexible material are aligned in the layer so that said portions are located on the same cross-section of the layer, i.e. on same level of the wall of the tubular occluding member. In some embodiments, the stiff layer may comprise four or more stiff-free areas, i.e. every complex stiff structure of the layer comprises four or more elementary stiff structures and three interconnected with three or more portions of a flexible material. Thus, the wall of a tubular occluding member may comprise along its longitudinal axis one, two or more ring-like areas (positioned perpendicularly to the longitudinal axis of the occluding member) that are free of the stiff material. Likewise, a solid occluding member made of a composite material comprising multiple complex stiff structures according to the invention has at least one stiff material free area along its longitudinal axis, wherein each such area corresponds to one cross-section of the occluding member which is perpendicular to the occluding member longitudinal axis. The width of the stiff material-free ring or thickness of the stiff material free-cross-section is defined by a portion of the interconnecting flexible material, i.e. its length within a complex stiff structure of the composite material; it may be from 1 to 50% of the total length of an occluding member, such as e.g. 5-25% of the total length.

Both a solid occluding member or the wall of a tubular occluding member may be made of a composite material that does not have a distinct layered structure described above, e.g. an occluding member may be made of a composite material wherein the stiff structures are embedded into the flexible material in parallel to each other fashion, but not organized to form a layer. The stiff structures of such composite material are preferably complex stiff structures; preferably, all complex stiff structures are represented the same type of a complex stiff structure; preferably, the complex stiff structures are aligned so that the portion(s) of the interconnecting flexible material of each complex stiff structure is (are) located in the same perpendicular cross-section to the longitudinal axis of the occluding member. Accordingly, a solid occluding member or the wall of a tubular occluding member made of such composite material comprises an area along its longitudinal axis that is free of the stiff material and consists of only the flexible material; preferably, there are at least two stiff material-free areas along the longitudinal axis of the solid or tubular occluding member made of such composite material.

In another embodiment, every stiff structure of the wall of a tubular occluding member or of a solid occluding member may be the elementary stiff structure e.g. may be represented by a single unbroken string, strip or plate, which comprise a hinge-like structure which may allow the stiff structure to bend, e.g. upon application of pressure to one or both ends of the stiff structure. According to the invention such hinge-like structure is equivalent to the interconnecting portion of a complex stiff structure. Accordingly, both tubular and solid occluding members made of a composite material comprising such elementary stiff structures are designed in the same way as tubular and solid occluding members made of a composite material comprising complex stiff structures, i.e. one or more hinge-like structures are located on the same level of the occluding member along its longitudinal axis.

In embodiments wherein the occluding member is made of a composite material comprising a framework of short stiff, e.g. pin-like, structures embedded into a flexible material, the composite material also comprises one or more areas which lack the stiff structures and consist of only flexible material. Said areas in a occluding member made of such composite material are located in the occluding member along its longitudinal axis in the same fashion as described above.

The areas of flexible material along an occluding member, i.e. along the longitudinal axis of an occluding member, forms one or more ring-like areas which are perpendicular to the longitudinal axis of the occluding member where the occluding member is capable to expand about its longitudinal axis upon application of pressure to the ends of the occluding member or upon application of another impact from a first smaller configuration that has transverse dimensions slightly smaller than the passageway of the body lumen where the occluding member is going to be introduced, to a second larger configuration that has transverse dimensions roughly corresponding to or slightly larger than the passageway of said body lumen, in order to secure the occluding member to the wall defining the body lumen. Such expanding portion of the occluding member is termed hereafter “expanding ring” or “expanding area”.

The expanding area may be positioned on any level of an occluding member along its longitudinal axis, e.g. at the ends or near the ends of the occluding member, around the middle part of the occluding member, etc. In one embodiment the occluding member comprises one expanding area along its longitudinal axis; in another embodiment, there are two expanding areas along the longitudinal axis of an occluding member; in some embodiments there may be tree or more expanding areas in a occluding member.

A great variety of different materials are suitable for preparation of a composite material of the invention. The flexible component may be prepared from any polymeric material, e.g. polyacrylates, polystyrene, polyethers, polytetrafluorethylene, polyvinylalcohol, polyethylene, and polypropylene; polymer blends, co-polymers, coated materials, etc. The stiff component, i.e. strings, strips or plates, may be prepared from the same polymeric material as the flexible compound or a different material. A polymeric material consisting of or conprising polyacrylates, polystyrenes, polyethers, polytetrafluorethylenes, polyvinylalcohols, polyethylenes and polypropylenes, polymer blends, co-polymers, coated materials, etc may be suitable as the stiff component. Alternatively, the strings, strips or plates may be metallic or comprise metals, such as nickel, titanium, silver, gold, platinum, palladium, steel, etc. and alloys thereof, or comprise thereof. Both the flexible and stiff material may comprise additives to strengthen or broaden their functional qualities. Preferably, the material of the flexible component or both flexible and stiff component is a biocompatible material. In one embodiment, the surface of an occluding member that faces the wall defining a reproductive tract may comprise copper or a copper alloy.

In one embodiment the flexible and/or stiff components consist of or comprise a shape memory material.

Expansion of the tubular occluding member from a primary configuration of relatively small transverse dimensions to a second larger configuration, once the occluding member is in place, may in one embodiment be achieved by heating the occluding member. In one embodiment, the wall of tubular occluding member may comprise flexible and/or stiff component made of a shape memory material has the ability to return from a deformed state (temporary shape) to its original (“remembered” permanent shape) state induced by an external stimulus (trigger), such as temperature change. By heating an elongated (temporary deformed shape) tubular occluding member comprising a shape-memory material to a temperature at or above the transition temperature of the shape-memory material, the occluding member may be transformed to a collapsed (permanent original shape) configuration which will cause the wall of the body lumen to be secured to the occluding member and the passageway through the body lumen to be occluded. In case if only one of two components of the composite material, i.e. either flexible or stiff component, is or comprises a shape memory material, the expansion of the tubular member will occur locally at places of the intersecting rings. The local expansion of the occluding member and corresponding local occlusion of the body lumen provided by the present invention may be in some embodiments advantageous, e.g. to reduce side effects of introduction of the occluding member into the body lumen, e.g. inflammation, or when the temporary occlusion is concerned: the occluding member secured to smaller portions of the wall defining the body lumen can be reduced with less effort and damages to the subject's body lumen.

Shape memory materials are well known in the art and have been successfully used in production of different medical devices (see e.g. Lendlein, A., Kelch, S. (2002). Shape-memory polymers. Angew. Chem. Int. Ed. 41: 2034-2057; Bellin, I., Kelch, S., Langer, R. & Lendlein, A. (2006). Polymeric triple-shape materials. Natl. Acad. Sci. U.S.A. 103: 18043-18; Lendlein, A. et al. (2005). Light-induced shape-memory polymers. Nature 434 (7035): 879-882; Sokolowski, W. et al (2007) Medical applications of shape memory polymers Biomed. Mater. 2 S23-S2; US 20090248141).

In one embodiment, the wall of a tubular occluding member may be made of a composite material that comprises a flexible component that may transversely expand upon application of pressure on the surface of said wall that defines the lumen of the occluding member, e.g. by filling the lumen of the occluding member with air, water, gel or otherwise, thereby securing the occluding member to a part of the wall defining the body lumen. The inflated flexible component may form a balloon-like structure, e.g. two balloon-like structures one in each end of the occluding member, wherein one may expand into uterus and the other toward the ovary end of tuba. The occluding member is in this way fixed and closes the passages through the tuba. The expansion may be limited in time so the balloon-like structures de-flats, e.g. after 1 day, 1 week, 1 month, 3 months or longer. The balloon-like structures may in one embodiment be filled with an active ingredient which may strengthen the function of the occluding member as sterilization device, e.g. with a spermatozoon toxic compound further preventing pregnancy.

In another embodiment, the flexible component may expand upon application pressure to the ends of the occlusion member, i.e. upon collapsing the occlusion member along its longitudinal axis.

An occluding member of the invention may be delivered to the desired location within the body lumen by any available means suitable for the purpose, e.g. by a conventional balloon catheter similar to those used for delivering stents, aortic grafts and various types of prosthesis.

In one embodiment, a device of the invention may comprise aids to facilitate sufficient securing the expanded occluding member to the wall of the body lumen to prevent the passage of fluid and cells therethrough, such as means stimulating endotherlialization of the occluding member within the body lumen.

In one embodiment a portion of the surface of the tubular occluding member facing the wall defining the body lumen may comprise nanostructures that are capable to promote growth and/or adhesion of endothelial cells so that the cells adhere to that portion of the surface of the occluding member and secure the occluding member in the lumen. For example, a portion of the surface of the occluding member may be nanostructured as e.g. described by Divia Rani V. V et al. (Nanotechnology 2009, 20(19): 195101), or by Pareta R A et al (Biotechnol. Bioeng. 2009; 103: 459-471).

In a preferred embodiment, portions of the surface of the wall of the tubular occluding member, or portions of the surface of a solid occluding member that comprise nanostructures promoting endothelial cell adhesion toward said surface are located on the same level along the longitudinal axis of the occluding member as the expanding ring-like areas. Accordingly, the nanostructured portions are arranged on the surface of the wall of the tubular occluding member as closed rings. The ring-like arrangement of the nanostructured portions that exactly corresponds to the areas of expansion of said member allows a precise and efficient securing the occluding member to the wall defining the body lumen.

In some embodiments, the surface of an occluding member facing the wall defining the body lumen may comprise one or more portions that comprise nanostructure(s) that can prevent the growth and/or adhesion of endothelial cells toward these portions. In a preferred embodiment, such nanostructured portions are also arranged as closed rings covering portions of the surface of an occluding member that are free of nanostructure(s) promoting cell adhesion. The occluding member having the surface nanostructured to prevent endotheliazation may be advantageous in some embodiments when a reverse occlusion of the body lumen is desired.

Nanostructured surfaces inhibiting cell adhesion and methods of producing thereof are also well-known in the art (see e.g. US 20080248031 or Kikuchi A., and Okano T. Nanostructured designs of biomedical materials: applications of cell sheet engineering to functional regenerative tissues and organs. J Controlled Release 2005, 101: 69-84).

Nanostructures examined for biointeraction may be engineered utilizing a number of conventional techniques. Examples of such techniques are described in Chen and Pepin, Electrophoresis (2001) 22:187-207; Marrian and Tnmnant, J. Vac. Sci. Technol., (2003) 21:207-215; and Gates et al., (2004) Annu. Rev. Mater. Res., 34:339-372.

Once the occluding member is inserted into the body lumen and, before or after, it has been sufficiently endothelialized to secure it to the body lumen wall, it may be expanded from the primary configuration to secondary configuration, e.g. by warming the occluding member to a temperature at or above the transition temperature of the shape-memory material so it may revert to its remember constricted shape, or by application of pleasure, or by other means. For example, the occluding member may be mounted onto the exterior of a balloon of a dilatation balloon catheter in the first configuration with small transverse dimensions, and then be introduced and positioned within the region of the body lumen to be occluded. The balloon is inflated to expand the occluding member, preferably with the outer diameter slightly larger than the inner dimensions of the body lumen to which it is secured. The occluding member will remain in the first configuration until heated to a temperature at or above its martensite to austenite transition temperature which causes it to revert to its collapsed state.

In one embodiment the device according to the invention is for reversible sterilisation of a female or male subject. Preferably, the occluding member of such device is non-surgically removable from the reproductive tract of the subject. In one preferred embodiment, the closings oft end 1 and end 2 of the occluding member may comprise a material that can be removed to allow opening the passage through the lumen of the occluding member. The material closing the ends of such occluding member may be a material that can be removed upon local application of heat, magnetic field, one or more chemical compounds or enzymes. Removing of the material may be remote-controlled.

The present invention provides effective sterilization or contraception for both males and females and importantly it is easily reversed. Moreover, the implantation and activation of the occluding member as well as the subsequent restoration of vessel patency requires easily used minimally invasive devices such as catheters, guidewires, guiding catheters and the like.

1. A composite material comprising at least two different components that differ from each other by their value of elastic modulus, wherein one component has a value of elastic modulus of less than 4 GPa, and another component has a value of elastic modulus of above 4 GPa, wherein the component that has a higher value of elastic modulus is embedded in the component that has a lower value of elastic modulus, said composite material is characterized in that it comprises at least one portion that is free of the component that has the higher value of elastic modulus, wherein said portion is capable of expanding in at least one direction upon application of pressure perpendicular to the expanding direction, or upon application of heat, light or a chemical or biological impact.

2. The composite material of claim 1, wherein the component that has a higher value of elastic modulus is shaped into pin-, string-, strip- or plate-like structures.

3. The composite material of claim 2, wherein a single structure is composed of two or more portions of the component that has a higher value of elastic modulus and one or more portions of the component that has a lower value of elastic modulus, wherein the portions of the component that has the lower value of elastic modulus interconnecting the portions of the component that has the higher value of elastic modulus.

4. The composite material according to claim 3, wherein the structures are embedded into the component having the lower value of elastic modulus so that said structures form one or more layers within said component, wherein said layers are separated with one or more layers of said component, each layer comprising a number of the same structures.

5. The composite material according to claim 4, wherein the structures are aligned within each layer so that each layer comprise a portion which is free of the component having the higher value of elastic modulus.

6. The composite material of claim 1, wherein the two different components consist of or comprise same polymeric material selected from polyacrylates, polystyrene, polyethers, polytetrafluorethylene, polyvinylalcohol, polyethylene, and polypropylene; polymer blends, co-polymers, coated materials.

7. The composite material of claim 1, wherein the two different components are different materials, wherein the component having the higher value of elastic modulus is a metal selected from nickel, gold, silver, titanium, steel or an alloy thereof, and component having the lower value of elastic modulus is a polymer selected from polyacrylates, polystyrene, polyethers, polytetrafluorethylene, polyvinylalcohol, polyethylene, polypropylene; polymer blends, co-polymers, coated materials.

8. The composite material of claim 1, wherein at least one of the components is a shape-memory material.

9. A device comprising an elongated cylinder-shaped member made of a composite material according to claim 1.

10. The device according to claim 9, wherein the member comprises a portion that is expandable about its longitudinal axis from a first smaller configuration to a second larger configuration upon application pressure or heat on said member.

11. The device according to claim 10, wherein at least one portion of the surface of the member is nanostructured.

12. The device according to claim 11, wherein the nanostructured portion of the surface of the member corresponds to the expandable portion of the member.

13. The device according to claim 9, wherein the device is an implantable contraceptive device for occluding the lumen of the fallopian tube of a female subject.

14. The device according to claim 13, wherein the device in the expanded configuration occludes the fallopian tube.

15. The device according to claim 11, wherein the nanostructured portion is capable of promoting growth and/or adhesion of endothelial cells of the wall of the lumen of the fallopian tube toward said portion.

16. The device according to claim 15, wherein the surface of the member comprises portions that comprise nanostructure(s) that prevent growth and/or adhesion of endothelial cells of the wall of the lumen of the fallopian tube toward said portions.

17. The device according to claim 12, wherein the member comprises a passageway along its longitudinal axis, and wherein said passageway is closed at both ends of the member.

18. The device according to 17, wherein the closures at the ends of the member are made of a material that is removable upon local application of heat, light, electric or magnetic field, or a chemical or biological impact.

19. The device according to claim 18, wherein the device is for reversible contraception.
